(19) United States
(12) Patent Application Publication
(10) Pub. No.: US 2009/0149925 A1
(43) Pub. Date: Jun. 11, 2009

(54) Temperature Indicator for Warming Products

(75) Inventors: J. Gavin MacDonald, Decatur, GA (US); Kaiyuan Yang, Cumming, GA (US); Kelly D. Arehart, Roswell, GA (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC., Neenah, WI (US)

(21) Appl. No.: 11/950,806

(22) Filed: Dec. 5, 2007

(51) Int. Cl. A61F 7/08; G01N 31/22

(52) U.S. Cl. 607/96; 436/7

(57) ABSTRACT

A warming product (e.g., mask, glove, sock, etc.) configured to provide heat to the body part of a user is provided. The warming product contains a thermochromic composition that undergoes a color change at a certain temperature. The color change may signal to a user that the warming product is hot, thus providing an indication that the desired treatment is still functioning. Likewise, the color change may signal that the product is cool, thus providing an indication that the treatment is complete.

### BACKGROUND OF THE INVENTION

Heat therapy is applied in a wide variety of contexts to reduce injury and to aid in recovery after exertions, injuries, and medical procedures. For example, heat therapy is often used for chronic conditions to help relax and loosen tissues, and to stimulate blood flow to the area. Heat treatments are also used for chronic conditions, such as overuse injuries, before participating in activities. A variety of electrical heating pads are known for providing heat therapy that are linked to a power cord that plugs into a wall outlet. However, such electrical pads are often undesirable because they are intimately linked with the location of an electrical outlet. Consequently, a variety of chemical warming packs have been developed that generate heat through an exothermic reaction. For example, chemical packs that generate heat via the reduction-oxidation of iron with oxygen can achieve a constant rate of heat generation by metering the amount of oxygen available to react with the iron. The duration of heat generation depends on the amount of iron available and the rate at which oxygen is made available to react with the iron. Unfortunately, however, it is often difficult to readily detect when the pack is warm enough to begin treatment, and conversely when the pack begins to cool near the completion of treatment.

As such, a need currently exists for a technique for simply and rapidly detecting the temperature of a warming product.

### SUMMARY OF THE INVENTION

In accordance with one embodiment of the present invention, a warming product for providing heat treatment to the body part of a user is disclosed. The warming product comprises an exothermic composition that releases heat for a certain time period upon activation, the time period defining a heating cycle. The warming product further comprises a thermochromic composition that possesses a first color during the heating cycle and a second color after completion of the heating cycle, the first color being visually distinguishable from the second color.

In accordance with another embodiment of the present invention, a method for monitoring the degree of heat being provided to a body part of a user is disclosed. The method comprises providing a warming product that comprises an exothermic composition that releases heat for a certain time period upon activation, the time period defining a heating cycle. The warming product further comprising a thermochromic composition that possesses a first color during the heating cycle and a second color after completion of the heating cycle. The exothermic composition is activated and the warming product is placed adjacent to a body part. The thermochromic composition is observed, wherein observation of the first color indicates that the exothermic composition is releasing heat during the heating cycle and observation of the second color indicates that the heating cycle is complete.

Other features and aspects of the present invention are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a perspective view of one embodiment of a warming product of the present invention;

FIG. 2 is a plan view of the warming product illustrated in FIG. 1;

FIG. 3 is a side view of the warming product illustrated in FIG. 2; and

FIG. 4 is a schematic illustration of another embodiment of a warming product of the present invention.

Repeat use of reference characters in the present specification and drawings is intended to represent same or analogous features or elements of the present disclosure.

### DETAILED DESCRIPTION OF REPRESENTATIVE EMBODIMENTS

As used herein the term “nonwoven” web or layer means a web having a structure of individual fibers or threads which are interlaid, but not in an identifiable manner as in a knitted fabric. Nonwoven webs may include, for instance, meltblown webs, spunbond webs, airlaid webs, carded webs, hydraulically entangled webs, etc. The basis weight of a nonwoven web may vary, such as from about 5 grams per square meter (“gsm”) to 150 gsm, in some embodiments from about 10 gsm to about 1000 gsm, and in some embodiments, from about 15 gsm to about 70 gsm.

As used herein, the term “meltblown web” generally refers to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Buntin, et al., which is incorporated herein in its entirety by reference thereto for all purposes. Generally speaking, meltblown fibers may be microfibers that are substantially continuous or discontinuous, generally smaller than 10 micrometers in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 micrometers, and are often between about 5 to about 20 micrometers.

As used herein, the term “coform” generally refers to a thermal composite material that contains a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic and/or organic absorbent materials, treated polymeric staple fibers and so forth. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georqer, et al.; which are incorporated herein in their entirety by reference thereto for all purposes.

### DETAILED DESCRIPTION

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

Generally speaking, the present invention is directed to a warming product (e.g., mask, glove, sock, etc.) configured to provide heat to the body part of a user. The warming product contains a thermochromic composition that undergoes a color change at a certain temperature. The color change may signal to a user that the warming product is hot, thus providing an indication that the desired treatment is still functioning. Likewise, the color change may signal that the product is cool, thus providing an indication that the treatment is complete.

Any thermochromic substance may generally be employed in the present invention. For example, liquid crystals may be employed as a thermochromic substance in some embodiments. The wavelength of light (“color”) reflected by liquid crystals depends in part on the pitch of the helical structure of the liquid crystal molecules. Because the length of this pitch varies with temperature, the color of the liquid crystals is also a function of temperature. One particular type of liquid crystal that may be used in the present invention is a liquid crystal cholesterol derivative. Exemplary liquid crystal cholesterol derivatives may include alkanoic and aralkanoic acid esters of cholesterol, alkyl esters of cholesterol carbonate, cholesterol chloride, cholesterol bromide, cholesterol acetate, cholesterol oleate, cholesterol caprylate, cholesterol oleyl-carbonate, and so forth. Other suitable liquid crystal cholesterol derivatives are described in U.S. Pat. No. 3,600,060 to Churchill, et al.; U.S. Pat. No. 3,619,254 to Davis; and U.S. Pat. No. 4,022,706 to Davis, which are incorporated herein in their entirety by reference thereto for all purposes.

In addition to liquid crystals, another suitable thermochromic substance that may be employed in the present invention is a proton accepting chromogen (“Lewis base”). In solution, the protonated form of the chromogen predominates at acidic pH levels (e.g., pH of about 4 or less). When the solution is made more alkaline through protonation, however, a color change occurs. One particularly suitable class of proton-accepting chromogens are leuco dyes, such as phthalides; phthalanes; acyl-leucomethylene compounds; fluoranes; spiropyranes; cumarins; and so forth. Exemplary fluoranes include, for instance, 3,3′-dimethoxyfluorane, 3,6-dimethoxyfluorane, 3,6-di-butoxyfluorane, 3-chloro-6-phenylamino-flourane, 3-diethylamino-6-dimethylfluorane, 3-diethylamino-6-methyl-7-chlorofluorane, and 3-diethyl-7,8-benzofluorane, 3,3′-bis-(p-dimethyl-aminophenyl)-7-phenylaminofluorane, 3-diethylamino-6-methyl-7-phenylamino-fluorane, 3-diethylamino-7-phenyl-aminofluorane, and 2-anilino-3-methyl-6-diethylamino-fluorane. Likewise, exemplary phthalides include 3,3′,3″-tris(p-dimethylamino-phenyl)phthalide, 3,3′-bis(p-dimethyl-aminophenyl)phthalide, 3,3-bis (p-diethylamino-phenyl)-6-dimethylamino-phthalide, 3-(4-diethylaminophenyl)-3-(1-ethyl-2-methylindol-3-yl)phthalide, and 3-(4-diethylamino-2-methyl)phenyl-3-(1,2-dimethylindol-3-yl)phthalide. Still other suitable chromogens are described in U.S. Pat. No. 4,620,941 to Yoshikawa, et al.; U.S. Pat. No. 5,281,570 to Hasegawa, et al.; U.S. Pat. No. 5,350,634 to Sumii, et al.; and U.S. Pat. No. 5,527,385 to Sumii, et al., which are incorporated herein in there entirety for all purposes.

A desensitizer may also be employed in conjunction with the proton-accepting chromogen to facilitate protonation at the desired temperature. More specifically, at a temperature below the melting point of the desensitizer, the chromogen generally possesses a first color (e.g., white). When the desensitizer is heated to its melting temperature, the chromogen becomes protonated, thereby resulting in a shift of the absorption maxima of the chromogen towards either the red (“bathochromic shift”) or blue end of the spectrum (“hypsochromic shift”). The nature of the color change depends on a variety of factors, including the type of proton-accepting chromogen utilized and the presence of any additional temperature-insensitive chromogens. The color change is typically reversible in that the chromogen deprotonates when cooled. Although any desensitizer may generally be employed in the present invention, it is typically desired that the desensitizer have a low volatility. For example, the desensitizer may have a boiling point of about 150° C. or higher, and in some embodiments, from about 170° C. to 280° C. Likewise, the melting temperature of the desensitizer is also typically from about 30° C. to about 60° C., in some embodiments from about 32° C. to about 55° C., and in some embodiments from about 34° C. to about 50° C. Examples of suitable desensitizers may include saturated or unsaturated alcohols containing about 6 to 30 carbon atoms, such as octyl alcohol, dodecyl alcohol, lauryl alcohol, cetyl alcohol, myristyl alcohol, stearyl alcohol, behenyl alcohol, geraniol, etc.; esters of saturated or unsaturated alcohols containing about 6 to 30 carbon atoms, such as butyl stearate, lauryl laurate, lauryl stearate, stearyl laurate, methyl myristate, decyl myristate, lauryl myristate, butyl stearate, lauryl palmitate, decyl palmitate, palmitic acid glyceride, etc.; azomethines, such as benzylideneaniline, benzylidenelaurylamide, o-methoxybenzylidene laurylamine, benzylidene p-toluidine, p-cumylbenzylidene, etc.; amides, such as acetamide, stearamide, etc.; and so forth.

The thermochromic composition may also include a proton-donating agent (also referred to as a “color developer”) to facilitate the reversibility of the color change. Such proton-donating agents may include, for instance, phenols, azoles, organic acids, esters of organic acids, and salts of organic acids. Exemplary phenols may include phenylphenol, bisphenol A, cresol, resorcinol, chlorolucinol, β-naphthol, 1,5-dihydroxynaphthalene, pyrocatechol, pyrogallol, trimer of p-chlorophenol-formaldehyde condensate, etc. Exemplary azoles may include benzotriaoles, such as 5-chlorobenzotriazole, 4-laurylaminosulfobenzotriazole, 5-butylbenzotriazole, dibenzotriazole, 2-oxybenzotriazole, 5-ethoxycarbonylbenzotriazole, etc.; imidazoles, such as oxybenzimidazole, etc.; tetrazoles; and so forth. Exemplary organic acids may include aromatic carboxylic acids, such as salicylic acid, methylenebissalicylic acid, resorcylic acid, gallic acid, benzoic acid, p-oxybenzoic acid, pyromellitic acid, β-naphthoic acid, tannic acid, toluic acid, trimellitic acid, phthalic acid, terephthalic acid, anthranilic acid, etc.; aliphatic carboxylic acids, such as stearic acid, 1,2-hydroxystearic acid, tartaric acid, citric acid, oxalic acid, lauric acid, etc.; and so forth. Exemplary esters may include alkyl esters of aromatic carboxylic acids in which the alkyl moiety has 1 to 6 carbon atoms, such as butyl gallate, ethyl p-hydroxybenzoate, methyl salicylate, etc.

If desired, one or more of the above-described components may be encapsulated to enhance the stability of the thermochromic substance during use. For example, a chromogen, desensitizer, developer, etc. may be mixed with a polymer resin (e.g., thermoset) according to any conventional method, such as interfacial polymerization, in-situ polymerization, etc. Suitable thermoset resins may include, for example, polyester resins, polyurethane resins, melamine resins, epoxy resins, diallyl phthalate resins, vinylester resins, and so forth. The resulting mixture may then be granulated and optionally coated with a hydrophilic macromolecular compound, such as alginic acid and salts thereof, carrageenan, pectin, gelatin and the like, semisynthetic macromolecular compounds such as methylcellulose, cationized starch, carboxymethylcellulose, carboxymethylated starch, vinyl polymers (e.g., polyvinyl alcohol), polyvinylpyrrolidone, polyacrylic acid, polyacrylamide, maleic acid copolymers, and so forth. The resulting microcapsules typically have a mean particle size of from about 5 nanometers to about 25 micrometers, in some embodiments from about 10 nanometers to about 10 micrometers, and in some embodiments, from about 50 nanometers to about 5 micrometers. Various other suitable encapsulation techniques are also described in U.S. Patent No. 4,957,949 to Kamada, et al.; U.S. Pat. No. 5,431,697 to Kamata, et al.; and U.S. Pat. No. 6,863,720 to Kitagawa, et al., which are incorporated herein in their entirety by reference thereto for all purposes.

The amount of the polymer resin(s) (e.g., thermoset) used to form such color-changing microcapsules may vary, but is typically from about 20 wt. % to about 80 wt. %, in some embodiments from about 30 wt. % to about 70 wt. %, and in some embodiments, from about 40 wt. % to about 60 wt. % of the microcapsules. The amount of the proton-accepting chromogen(s) employed may be from about 0.1 wt. % to about 20 wt. %, in some embodiments from about 0.5 wt. % to about 15 wt. %, and in some embodiments, from about 1 to about 10 wt. % of the microcapsules. The proton-donating agent(s) may constitute from about 0.5 to about 30 wt. %, in some embodiments from about 1 wt. % to about 20 wt. %, and in some embodiments, from about 2 wt. % to about 15 wt. % of the microcapsules. In addition, the desensitizer(s) may constitute from about 10 wt. % to about 70 wt. %, in some embodiments from about 15 wt. % to about 60 wt. %, and in some embodiments, from about 20 wt. % to about 50 wt. % of the microcapsules.

The nature and weight percentage of the components used in the color-changing composition are generally selected so that it changes from one color to another color, from no color to a color, or from a color to no color at a desired activation temperature. The desired activation temperature depends largely on the specific nature of the warming product. Warming products, for example, are generally designed to reach an elevated temperature of from about 30° C. to about 60° C., in some embodiments from about 32° C. to about 55° C., and in some embodiments from about 34° C. to about 50° C. Thus, the thermochromic composition may have an activation temperature within the ranges noted above so that it possesses one color when the warming product is providing heat, and another color when the heat is exhausted and the product begins to cool. Commercially available thermochromic substances that have an activation temperature with the desired ranges may be obtained from Matsui Shikiso Chemical Co., Ltd. of Kyoto, Japan under the designation “Chromicolor” (e.g., Chromicolor AQ-Ink) or from Color Change Corporation of Streamwood, Ill. (e.g., black leuco powder having a transition of 33° C. or 41° C., blue leuco powder having a transition of 33° C. or 36° C., etc.).

The thermochromic composition of the present invention is applied to the warming product so that it is visible during use. For example, the composition may be coated onto one or more surfaces of a substrate (e.g., nonwoven web, woven fabric, knit fabric, paper web, film, foam, etc.) of the warming product using any known technique, such as printing, dipping, spraying, melt extruding, coating (e.g., solvent coating, powder coating, brush coating, etc.), and so forth. The thermochromic composition may cover an entire surface of the warming product, or may only cover a portion of the surface. For instance, to maintain absorbency, porosity, flexibility, and/or some other characteristic of the warming product, it may sometimes be desired to apply the thermochromic composition so as to cover less than 100%, in some embodiments from about 10% to about 80%, and in some embodiments, from about 20% to about 60% of the area of one or more surfaces of the warming product. The thermochromic composition may, for example, be applied to the warming product in a preselected pattern (e.g., reticular pattern, diamond-shaped grid, dots, and so forth). It should be understood, however, that the coating may also be applied uniformly to one or more surfaces of the warming product.

If desired, the thermochromic composition may also be applied to a separate substrate (e.g., strip) that is subsequently adhered or otherwise attached to the warming product. For example, the strip may contain a facestock material commonly employed in the manufacture of labels, such as paper, polyester, polyethylene, polypropylene, polybutylene, polyamides, etc. An adhesive, such as a pressure-sensitive adhesive, heat-activated adhesive, hot melt adhesive, etc., may be employed on one or more surfaces of the facestock material to help adhere it to a surface of the substrate. Suitable examples of pressure-sensitive adhesives include, for instance, acrylic-based adhesives and elastomeric adhesives. In one embodiment, the pressure-sensitive adhesive is based on copolymers of acrylic acid esters (e.g., 2-ethyl hexyl acrylate) with polar co-monomers (e.g., acrylic acid). The adhesive may have a thickness in the range of from about 0.1 to about 2 mils (2.5 to 50 microns). A release liner may also be employed that contacts the adhesive prior to use. The release liner may contain any of a variety of materials known to those of skill in the art, such as a silicone-coated paper or film substrate.

In addition to being coated onto the warming product, the thermochromic composition may also be incorporated into one or more substrates of the warming product. For example, the thermochromic composition may be compounded with a melt-extrudable thermoplastic composition to form a film, fiber, or nonwoven web used in the warming product. In such embodiments, the thermochromic composition may be pre-blended with a carrier resin to form a masterbatch that is compatible with the thermoplastic composition. Because the thermochromic composition may be more miscible with amorphous regions of a polymer than the crystalline regions, the carrier resin may be generally amorphous or semi-crystalline to optimize compatibility. Exemplary amorphous polymers include polystyrene, polycarbonate, acrylic, acrylonitrile-butadiene-styrene (ABS), styrene-acrylonitrile, and polysulfone. Exemplary semi-crystalline polymers include high and low density polyethylene, polypropylene, polyoxymethylene, poly(vinylidine fluoride), poly(methyl pentene), poly(ethylene-chlorotrifluoroethylene), poly(vinyl fluoride), poly(ethylene oxide), poly(ethylene terephthalate), poly(butylene terephthalate), nylon 6, nylon 66, poly(vinyl alcohol) and polybutene. Particularly desired semi-crystalline polymers are predominantly linear polymers having a regular structure. Examples of semi-crystalline, linear polymers that may be used in the present invention include polyethylene, polypropylene, blends of such polymers and copolymers of such polymers. Semi-crystalline polyethylene-based polymers, for instance, may have a melt index of greater than about 5 grams per 10 minutes, and in some embodiments, greater than about 10 grams per 10 minutes (Condition E at 190° C., 2.16 kg), as well as a density of greater than about 0.910 grams per cubic centimeter (g/cm³), in some embodiments greater than about 0.915 g/cm³, in some embodiments from about 0.915 to about 0.960 g/cm³, in some embodiments from about 0.917 and 0.960 g/cm³. Likewise, semi-crystalline polypropylene-based polymers may have a melt index of greater than about 10 grams per 10 minutes, and in some embodiments, greater than about 20 grams per 10 minutes, as well as a density of from about 0.89 to about 0.90 g/cm³. Specific examples of such polymers include ExxonMobil 3155, Dow polyethylenes such as DOWLEX™ 2517; Dow LLDPE DNDA-1082, Dow LLDPE DNDB-1077, Dow LLDPE 1081, and Dow LLDPE DNDA 7147. In some instances, higher density polymers may be useful, such as Dow HDPE DMDA-8980. Additional resins include Escorene™ LL 5100 and Escorene™ LL 6201 from ExxonMobil. Polypropylene-based resins having a density of from about 0.89 g/cm³ to about 0.90 g/cm³ may also be used, such as homopolymers and random copolymers such as ExxonMobil PP3155, PP1074KN, PP9074MED and Dow 6D43.

The amount of the carrier resin employed will generally depend on a variety of factors, such as the type of carrier resin and thermoplastic composition, the processing conditions, etc. Typically, the carrier resin constitutes from about 10 wt. % to about 80 wt. %, in some embodiments from about 20 wt. % to about 70 wt. %, and in some embodiments, from about 40 wt. % to about 60 wt. % of the masterbatch. The thermochromic substance likewise normally constitutes from about 10 wt. % to about 80 wt. %, in some embodiments from about 20 wt. % to about 70 wt. %, and in some embodiments, from about 40 wt. % to about 60 wt. % of the masterbatch.

The carrier resin may be blended with the thermochromic substance using any known technique, such as batch and/or continuous compounding techniques that employ, for example, a Banbury mixer, Farrel continuous mixer, single screw extruder, twin screw extruder, etc. If desired, the carrier resin and thermochromic substance may be dry blended, i.e., without a solvent. After blending, the masterbatch may be processed immediately or compression molded into pellets for subsequent use. One suitable compression molding device is a die and roller type pellet mill. Specifically, the masterbatch (in granular form) is fed continuously to a pelletizing cavity. The masterbatch is compressed between a die and rollers of the cavity and forced through holes in the die. As pellets of the composition are extruded, a knife or other suitable cutting surface may shear the pellets into the desired size.

Regardless of whether the thermochromic substance is pre-blended with a carrier resin, it may be ultimately compounded with a melt-extrudable thermoplastic composition to form a substrate (e.g., film, fiber, or nonwoven web). The thermochromic substance or masterbatch containing the substance may be miscible with the thermoplastic composition. Otherwise, the components may simply be blended under high shear or modified to improve their interfacial properties. The thermochromic substance may be blended with the thermoplastic composition (e.g., polypropylene or polyethylene) before melt extrusion or within the extrusion apparatus itself. The thermochromic substance may constitute from about 0.001 wt. % to about 10 wt. %, in some embodiments from about 0.01 wt. % to about 5 wt. %, and in some embodiments, from about 0.1 wt. % to about 1 wt. % of the blend.

Exemplary melt-extrudable polymers suitable for use in the thermoplastic composition may include, for example, polyolefins, polyesters, polyamides, polycarbonates, copolymers and blends thereof, etc. Suitable polyolefins include polyethylene, such as high density polyethylene, medium density polyethylene, low density polyethylene, and linear low density polyethylene; polypropylene, such as isotactic polypropylene, atactic polypropylene, and syndiotactic polypropylene; polybutylene, such as poly(1-butene) and poly(2-butene); polypentene, such as poly(1-pentene) and poly(2-pentene); poly(3-methyl-1-pentene); poly(4-methyl-1-pentene); and copolymers and blends thereof. Suitable copolymers include random and block copolymers prepared from two or more different unsaturated olefin monomers, such as ethylene/propylene and ethylene/butylene copolymers. Suitable polyamides include nylon 6, nylon 6/6, nylon 4/6, nylon 11, nylon 12, nylon 6/10, nylon 6/12, nylon 12/12, copolymers of caprolactam and alkylene oxide diamine, etc., as well as blends and copolymers thereof. Suitable polyesters include poly(lactide) and poly(lactic acid) polymers as well as polyethylene terephthalate, polybutylene terephthalate, polytetramethylene terephthalate, polycyclohexylene-1,4-dimethylene terephthalate, and isophthalate copolymers thereof, as well as blends thereof.

If desired, elastomeric polymers may also be used in the thermoplastic composition, such as elastomeric polyesters, elastomeric polyurethanes, elastomeric polyamides, elastomeric polyolefins, elastomeric copolymers, and so forth. Examples of elastomeric copolymers include block copolymers having the general formula A-B-A′ or A-B, wherein A and A′ are each a thermoplastic polymer endblock that contains a styrenic moiety and B is an elastomeric polymer midblock, such as a conjugated diene or a lower alkene polymer. Such copolymers may include, for instance, styrene-isoprene-styrene (S-I-S), styrene-butadiene-styrene (S-B-S), styrene-ethylene-butylene-styrene (S-EB-S), styrene-isoprene (S-I), styrene-butadiene (S-B), and so forth. Commercially available A-B-A′ and A-B-A-B copolymers include several different S-EB-S formulations from Kraton Polymers of Houston, Tex. under the trade designation KRATON®. KRATON® block copolymers are available in several different formulations, a number of which are identified in U.S. Patent Nos. 4,663,220, 4,323,534, 4,834,738, 5,093,422 and 5,304,599, which are hereby incorporated in their entirety by reference thereto for all purposes. Other commercially available block copolymers include the S-EP-S elastomeric copolymers available from Kuraray Company, Ltd. of Okayama, Japan, under the trade designation SEPTON®. Still other suitable copolymers include the S-I-S and S-B-S elastomeric copolymers available from Dexco Polymers of Houston, Tex. under the trade designation VECTOR®. Also suitable are polymers composed of an A-B-A-B tetrablock copolymer, such as discussed in U.S. Patent No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer.

Examples of elastomeric polyolefins include ultra-low density elastomeric polypropylenes and polyethylenes, such as those produced by “single-site” or “metallocene” catalysis methods. Such elastomeric olefin polymers are commercially available from ExxonMobil Chemical Co. of Houston, Tex. under the trade designations ACHIEVE® (propylene-based), EXACT® (ethylene-based), and EXCEED® (ethylene-based). Elastomeric olefin polymers are also commercially available from DuPont Dow Elastomers, LLC (a joint venture between DuPont and the Dow Chemical Co.) under the trade designation ENGAGE® (ethylene-based) and from Dow Chemical Co. of Midland, Mich. under the name AFFINITY® (ethylene-based). Examples of such polymers are also described in U.S. Pat. Nos. 5,278,272 and 5,272,236 to Lai, et al., which are incorporated herein in their entirety by reference thereto for all purposes. Also useful are certain elastomeric polypropylenes, such as described in U.S. Pat. No. 5,539,056 to Yang, et al. and U.S. Pat. No. 5,596,052 to Resconi, et al., which are incorporated herein in their entirety by reference thereto for all purposes.

Once formed, the resulting melt-extrudable blend may then be extruded through a die. Although the die may have any desired configuration, it typically contains a plurality of orifices arranged in one or more rows extending the full width of the machine. The orifices may be circular or noncircular in cross-section. As stated above, the extruded blend may be formed into a thermochromic film in some embodiments of the present invention. Any known technique may be used to form a film from the thermochromic substance, including blowing, casting, flat die extruding, etc. For example, a thermochromic film may be formed by melt extruding the blend, immediately chilling the extruded material (e.g., on a chilled roll) to form a precursor film, and optionally orienting the precursor film in the machine direction, cross machine direction, or both. Alternatively, thermochromic fibers may also be formed according to the present invention. Such fibers may be formed by melt extruding the blend, attenuating the extruded material, and collecting the fibers on a roll (e.g., godet roll) for direct use or on a moving foraminous surface to form a thermochromic nonwoven web.

The thermochromic composition typically constitutes from about 0.5 wt. % to about 25 wt. %, in some embodiments from about 1 wt. % to about 20 wt. %, and in some embodiments, from about 2 wt. % to about 10 wt. % of the dry weight of the warming product. Of course, the actual amount may vary based on a variety of factors, including the nature of the substrate, sensitivity of the thermochromic substance, the presence of other additives, the desired degree of detectability (e.g., with an unaided eye), etc.

To provide heat to the desired body part, the warming product of the present invention generally contains an exothermic composition that is capable of generating heat upon activation. The components of the composition may release heat through a physical process, chemical reaction, etc. Reactants that may undergo an exothermic reaction include, for instance, quick lime, sodium hydroxide, cobalt, chromium, iron hydroxide, magnesium, manganese, molybdenum, tin oxide(II), titanium, sodium, sodium acetate crystals, calcium hydroxide, metallic sodium, magnesium chloride, anhydrous calcium chloride (CaCl₂), sodium thiosulfate, and the hydration of zeolites (e.g. sodium aluminosilicates). Other suitable reactants are believed to be described in U.S. Pat. No. 5,792,213 to Bowen and U.S. Pat. No. 6,248,125 to Helming, which are incorporated herein in their entirety by reference thereto for all purposes.

In one particular embodiment, the exothermic composition includes an oxidizable metal that is capable of releasing heat in the presence of air and optionally moisture. Examples of such metals include, but are not limited to, iron, zinc, aluminum, magnesium, nickel, etc., as well as metal oxides or hydroxides (e.g., manganese hydroxide). Although not required, the metal may be initially provided in powder form to facilitate handling and to reduce costs. Various methods for removing impurities from a crude metal (e.g., iron) to form a powder include, for example, wet processing techniques, such as solvent extraction, ion exchange, and electrolytic refining for separation of metallic elements; hydrogen gas (H₂) processing for removal of gaseous elements, such as oxygen and nitrogen; floating zone melting refining method. Using such techniques, the metal purity may be at least about 95%, in some embodiments at least about 97%, and in some embodiments, at least about 99%. The particle size of the metal powder may also be less than about 500 micrometers, in some embodiments less than about 100 micrometers, and in some embodiments, less than about 50 micrometers. The use of such small particles may enhance the contact surface of the metal with air, thereby improving the likelihood and efficiency of the desired exothermal reaction. The concentration of the metal powder employed may generally vary depending on the nature of the metal powder, and the desired extent of the exothermal/oxidation reaction. In most embodiments, the metal powder is present in the exothermic composition in an amount from about 40 wt. % to about 95 wt. %, in some embodiments from about 50 wt. % to about 90 wt. %, and in some embodiments, from about 60 wt. % to about 80 wt. %.

In addition to an oxidizable metal, a carbon component may also be utilized in the exothermic composition. It is believed that such a carbon component promotes the oxidation reaction of the metal and acts as a catalyst for generating heat. The carbon component may be activated carbon, carbon black, graphite, and so forth. When utilized, activated carbon may be formed from sawdust, wood, charcoal, peat, lignite, bituminous coal, coconut shells, etc. Some suitable forms of activated carbon and techniques for formation thereof are described in U.S. Pat. No. 5,693,385 to Parks; U.S. Pat. No. 5,834,114 to Economy, et al.; U.S. Pat. No. 6,517,906 to Economy, et al.; U.S. Pat. No. 6,573,212 to McCrae, et al., as well as U.S. Patent Application Publication Nos. 2002/0141961 to Falat, et al. and 2004/0166248 to Hu, et al., all of which are incorporated herein in their entirety by reference thereto for all purposes.

The exothermic composition may also employ a binder for enhancing the durability of the exothermic composition when applied to the warming product. Any of a variety of binders may be used in the exothermic composition of the present invention. Suitable binders may include, for instance, those that become insoluble in water upon crosslinking. Crosslinking may be achieved in a variety of ways, including by reaction of the binder with a polyfunctional crosslinking agent. Examples of such crosslinking agents include, but are not limited to, dimethylol urea melamine-formaldehyde, urea-formaldehyde, polyamide epichlorohydrin, etc. In some embodiments, a polymer latex may be employed as the binder. The polymer suitable for use in the latexes typically has a glass transition temperature of about 30° C. or less so that the flexibility of the resulting warming product is not substantially restricted. Moreover, the polymer also typically has a glass transition temperature of about −25° C. or more to minimize the tackiness of the polymer latex. For instance, in some embodiments, the polymer has a glass transition temperature from about −15° C. to about 15° C., and in some embodiments, from about −10° C. to about 0° C. For instance, some suitable polymer latexes that may be utilized in the present invention may be based on polymers such as, but are not limited to, styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, nitrile polymers, and any other suitable anionic polymer latex polymers known in the art. The charge of the polymer latexes described above may be readily varied, as is well known in the art, by utilizing a stabilizing agent having the desired charge during preparation of the polymer latex. Specific carbon/polymer latex systems are described in more detail in U.S. Patent Nos. 6,573,212; 6,639,004; 5,693,385; and 5,540,916. Activated carbon/polymer latex systems that may be used in the present invention include Nuchar® PMA, DPX-8433-68A, and DPX-8433-68B, all of which are made by MeadWestvaco Corp of Stamford, Conn.

If desired, the polymer latex may be crosslinked using any known technique in the art, such as by heating, ionization, etc. Preferably, the polymer latex is self-crosslinking in that external crosslinking agents (e.g., N-methylol acrylamide) are not required to induce crosslinking. Specifically, crosslinking agents may lead to the formation of bonds between the polymer latex and the warming product to which it is applied. Such bonding may sometimes interfere with the effectiveness of the warming product in generating heat. Thus, the polymer latex may be substantially free of crosslinking agents. Particularly suitable self-crosslinking polymer latexes are ethylene-vinyl acetate copolymers available from Celanese Corp. of Dallas, Tex. under the designation DUR-O-SET® Elite (e.g., PE-25220A, PE-LV 25-432A). Alternatively, an inhibitor may simply be employed that reduces the extent of crosslinking, such as free radical scavengers, methyl hydroquinone, t-butylcatechol, pH control agents (e.g., potassium hydroxide), etc.

Although polymer latexes may be effectively used as binders in the present invention, such compounds sometimes result in a reduction in drapability and an increase in residual odor. Thus, water-soluble organic polymers may also be employed, either alone or in conjunction with the polymer latexes, to alleviate such concerns. For example, one class of water-soluble organic polymers found to be suitable in the present invention is polysaccharides and derivatives thereof (e.g., cellulosic ethers). Polysaccharides are polymers containing repeated carbohydrate units, which may be cationic, anionic, nonionic, and/or amphoteric. In one particular embodiment, for instance, the polysaccharide is a nonionic, cationic, anionic, and/or amphoteric cellulosic ether. Nonionic cellulose ethers, for instance, may be produced in any manner known to those skilled in the art, such as by reacting alkali cellulose with ethylene oxide and/or propylene oxide, followed by reaction with methyl chloride, ethyl chloride and/or propyl chloride. Nonionic cellulosic ethers and methods for producing such ethers are described, for instance, in U.S. Pat. No. 6,123,996 to Larsson, et al.; U.S. Pat. No. 6,248,880 to Karlson; and U.S. Pat. No. 6,639,066 to Bostrom, et al., which are incorporated herein in their entirety by reference thereto for all purposes. Some suitable examples of nonionic cellulosic ethers include, but are not limited to, water-soluble alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth. Preferred nonionic cellulosic ethers for use in the coating composition of the present invention are ethyl hydroxyethyl cellulose, methylethyl hydroxyethyl cellulose, methylethyl hydroxyethyl hydroxypropyl cellulose and methyl hydroxypropyl cellulose. In such embodiments, the hydroxyethyl groups typically constitute at least 30% of the total number of hydroxyalkyl groups, and the number of ethyl substituents typically constitutes at least 10% of the total number of alkyl substituents.

Particularly suitable cellulosic ethers may include, for instance, those available from Akzo Nobel of Stamford, Conn. under the name “BERMOCOLL.” Still other suitable cellulosic ethers are those available from Shin-Etsu Chemical Co., Ltd. of Tokyo, Japan under the name “METOLOSE”, including METOLOSE Type SM (methycellulose), METOLOSE Type SH (hydroxypropylmethyl cellulose), and METOLOSE Type SE (hydroxyethylmethyl cellulose). One particular example of a suitable nonionic cellulosic ether is methylcellulose having a degree of methoxyl substitution (DS) of 1.8. The degree of methoxyl substitution represents the average number of hydroxyl groups present on each anhydroglucose unit that have been reacted, which may vary between 0 and 3. One such cellulosic ether is METOLOSE SM-100, which is a methylcellulose commercially available from Shin-Etsu Chemical Co., Ltd. Other suitable cellulosic ethers are also available from Hercules, Inc. of Wilmington, Del. under the name “CULMINAL.” Further examples of suitable polysaccharides are described in more detail above.

The concentration of the carbon component and/or binder in the exothermic composition may generally vary based on the desired properties of the warming product. For example, the amount of the carbon component is generally tailored to facilitate the oxidation/exothermic reaction without adversely affecting other properties of the warming product. Typically, the carbon component is present in the exothermic composition in an amount about 0.01 wt. % to about 20 wt. %, in some embodiments from about 0.1 wt. % to about 15 wt. %, and in some embodiments, from about 1 wt. % to about 12 wt. %. In addition, although relatively high binder concentrations may provide better physical properties for the exothermic composition, they may likewise have an adverse effect on other properties, such as the absorptive capacity of the warming product to which it is applied. Conversely, relatively low binder concentrations may reduce the ability of the exothermic composition to remain affixed on the warming product. Thus, in most embodiments, the binder is present in the exothermic composition in an amount from about 0.01 wt. % to about 20 wt. %, in some embodiments from about 0.1 wt. % to about 10 wt. %, and in some embodiments, from about 0.5 wt. % to about 8 wt. %.

Still other components may also be employed in the exothermic composition of the present invention. For example, as is well known in the art, an electrolytic salt may be employed to react with and remove any passivating layer(s) that might otherwise prevent the metal from oxidizing. Suitable electrolytic salts may include, but are not limited to, alkali halides or sulfates, such as sodium chloride, potassium chloride, etc.; alkaline halides or sulfates, such as calcium chloride, magnesium chloride, etc., and so forth. When employed, the electrolytic salt is typically present in the exothermic composition in an amount from about 0.01 wt. % to about 10 wt. %, in some embodiments from about 0.1 wt. % to about 8 wt. %, and in some embodiments, from about 1 wt. % to about 6 wt. %.

In addition to the above-mentioned components, other components, such as surfactants, pH adjusters, dyes/pigments/inks, viscosity modifiers, moisture-retaining particles, etc., may also be included in the exothermic coating of the present invention. Viscosity modifiers may be used, for example, to adjust the viscosity of the coating formulation based on the desired coating process and/or performance of the coated warming product. Suitable viscosity modifiers may include gums, such as xanthan gum. Binders, such as the cellulosic ethers, may also function as suitable viscosity modifiers. When employed, such additional components typically constitute less than about 5 wt. %, in some embodiments less than about 2 wt. %, and in some embodiments, from about 0.001 wt. % to about 1 wt. % of the exothermic coating.

The exothermic composition may be incorporated into the warming product in a variety of ways. In certain embodiments, for example, the exothermic composition may be coated onto a substrate of the warming product, either alone or in conjunction with a thermochromic composition, using any conventional technique, such as bar, roll, knife, curtain, print (e.g., rotogravure), spray, slot-die, drop-coating, or dip-coating techniques. The solids add-on level of the exothermic composition may be varied as desired. The “solids add-on level” is determined by subtracting the weight of the untreated warming product from the weight of the treated warming product (after drying), dividing this calculated weight by the weight of the untreated warming product, and then multiplying by 100%. Lower add-on levels may optimize certain properties (e.g., absorbency), while higher add-on levels may optimize heat generation. In some embodiments, for example, the add-on level is from about 100% to about 5000%, in some embodiments from about 200% to about 2400%, and in some embodiments, from about 400% to about 1200%. The thickness of the exothermic composition may also vary. For example, the thickness may range from about 0.01 millimeters to about 5 millimeters, in some embodiments, from about 0.01 millimeters to about 3 millimeters, and in some embodiments, from about 0.1 millimeters to about 2 millimeters. In some cases, a relatively thin coating may be employed (e.g., from about 0.01 millimeters to about 0.5 millimeters). Such a thin coating may enhance the flexibility of the warming product, while still providing uniform heating.

When the exothermic composition is capable of activation in the presence of moisture and air, such as described above, it may be desired to initially heat the substrate to remove moisture from the exothermic composition prior to use. For example, the substrate may be heated to a temperature of at least about 100° C., in some embodiments at least about 110° C., and in some embodiments, at least about 120° C. In this manner, the resulting dried exothermic composition is anhydrous, i.e., generally free of water. By minimizing the amount of moisture, the exothermic composition is less likely to react prematurely and generate heat. Thus, the exothermic composition may remain inactive until placed in the vicinity of moisture. It should be understood, however, that relatively small amounts of water may still be present in the exothermic composition without causing a substantial exothermic reaction. In some embodiments, for example, the exothermic composition contains water in an amount less than about 0.5% by weight, in some embodiments less than about 0.1% by weight, and in some embodiments, less than about 0.01% by weight.

To activate the exothermic composition, moisture may be applied during the normal course of use or as an additional activation step. When applying moisture in an additional activation step, various techniques may be employed, including spraying, dipping, coating, dropping (e.g., using a syringe), etc. Likewise, moisture simply absorbed from the surrounding environment may activate the composition. In some cases, it may be desired to control the amount of moisture and air that contacts the exothermic composition to achieve a certain reaction rate. For example, it may be desired to limit the rate at which the exothermic reaction proceeds to prevent too great of a temperature increase. If desired, one or more components may be used in conjunction with the coated substrate to retain moisture and controllably transfer it to the substrate upon activation. In one embodiment, for example, a moisture-holding layer may be positioned near or adjacent to the substrate to absorb and hold moisture for an extended period of time.

The moisture-holding layer helps control the moisture application rate by holding moisture and controllably releasing it to the exothermic composition over an extended period of time. Thus, moisture may be supplied directly from the moisture-holding layer to the exothermic composition. The moisture-holding layer may contain an absorbent web formed using any technique, such as a dry-forming technique, an airlaying technique, a carding technique, a meltblown or spunbond technique, a wet-forming technique, a foam-forming technique, etc. Airlaid webs, for instance, are made from bundles of fibers having typical lengths ranging from about 3 to about 19 millimeters, which are separated, entrained in an air supply, and then deposited onto a forming surface, usually with the assistance of a vacuum supply. The randomly deposited fibers then are bonded to one another using, for example, hot air or an adhesive.

The moisture-holding layer typically contains cellulosic fibers, such as natural and/or synthetic fluff pulp fibers. The fluff pulp fibers may be kraft pulp, sulfite pulp, thermomechanical pulp, etc. In addition, the fluff pulp fibers may include high-average fiber length pulp, low-average fiber length pulp, or mixtures of the same. One example of suitable high-average length fluff pulp fibers includes softwood kraft pulp fibers. Softwood kraft pulp fibers are derived from coniferous trees and include pulp fibers such as, but not limited to, northern, western, and southern softwood species, including redwood, red cedar, hemlock, Douglas-fir, true firs, pine (e.g., southern pines), spruce (e.g., black spruce), combinations thereof, and so forth. Northern softwood kraft pulp fibers may be used in the present invention. One example of commercially available southern softwood kraft pulp fibers suitable for use in the present invention include those available from Weyerhaeuser Company with offices in Federal Way, Wash. under the trade designation of “NB-416.” Another type of fluff pulp that may be used in the present invention is identified with the trade designation CR1654, available from U.S. Alliance of Childersburg, Ala., and is a bleached, highly absorbent sulfate wood pulp containing primarily softwood fibers. Still another suitable fluff pulp for use in the present invention is a bleached, sulfate wood pulp containing primarily softwood fibers that is available from Bowater Corp. with offices in Greenville, S.C. under the trade name CoosAbsorb S pulp. Low-average length fibers may also be used in the present invention. An example of suitable low-average length pulp fibers is hardwood kraft pulp fibers. Hardwood kraft pulp fibers are derived from deciduous trees and include pulp fibers such as, but not limited to, eucalyptus, maple, birch, aspen, etc. Eucalyptus kraft pulp fibers may be particularly desired to increase softness, enhance brightness, increase opacity, and change the pore structure of the sheet to increase its wicking ability.

If desired, the moisture-holding layer may also contain synthetic fibers, such as monocomponent and multicomponent (e.g., bicomponent) fibers. The moisture-holding layer may also include a superabsorbent material, such as natural, synthetic and modified natural materials. Superabsorbent materials are water-swellable materials capable of absorbing at least about 20 times its weight and, in some cases, at least about 30 times its weight in an aqueous solution containing 0.9 weight percent sodium chloride. Examples of synthetic superabsorbent material polymers include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further superabsorbent materials include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and so forth. Mixtures of natural and wholly or partially synthetic superabsorbent polymers may also be useful in the present invention. Other suitable absorbent gelling materials are disclosed in U.S. Pat. No. 3,901,236 to Assarsson et al.; U.S. Pat. No. 4,076,663 to Masuda et al.; and U.S. Pat. No. 4,286,082 to Tsubakimoto et al., which are incorporated herein in their entirety by reference thereto for all purposes.

When utilized, the superabsorbent material may constitute from about 1 wt. % to about 40 wt. %, in some embodiments, from about 5 wt. % to about 30 wt. %, and in some embodiments, from about 10 wt. % to about 25 wt. % of the moisture-holding layer (on a dry basis). Likewise, synthetic fibers may constitute from about 1 wt. % to about 30 wt. %, in some embodiments, from about 2 wt. % to about 20 wt. %, and in some embodiments, from about 5 wt. % to about 15 wt. % of the moisture-holding layer (on a dry basis). The cellulosic fibers may also constitute up to 100 wt. %, in some embodiments from about 50 wt. % to about 95 wt. %, and in some embodiments, from about 65 wt. % to about 85 wt. % of the moisture-holding layer (on a dry basis).

Moisture (e.g., water) may be pre-applied to the moisture-holding layer any time prior to or during use of the warming product, such as during manufacture. The moisture is added in an amount effective to activate an exothermic, electrochemical reaction between the electrochemically oxidizable element (e.g., metal powder) and an electrochemically reducible element (e.g., oxygen). Although this amount may vary depending on the reaction conditions and the amount of heat desired, the moisture is typically added in an amount from about 20 wt. % to about 500 wt. %, and in some embodiments, from about 50 wt. % to about 200 wt. %, of the weight of the amount of oxidizable metal present in the coating. Although not necessarily required, it may be desired to seal such water-treated warming products within a substantially liquid-impermeable material and vapor-impermeable that inhibits the exothermic composition from contacting enough oxygen to prematurely activate the exothermic reaction. To generate heat, the warming product is simply removed from the package and exposed to air.

Although various embodiments of a warming product have been described above, it should be understood that other configurations are also included within the scope of the present invention. For instance, other layers may also be employed to improve the exothermic properties of the warming product. For example, multiple substrate may be coated with the exothermic composition and employed in the warming product. The substrates may function together to provide heat to a surface, or may each provide heat to different surfaces. In addition, substrates may be employed that are not applied with the exothermic composition of the present invention, but instead applied with a coating that simply facilitates the reactivity of the exothermic composition. Still other layers may also be employed in the warming product if desired. For example, the warming product may contain a liquid-impermeable and vapor-permeable (“breathable”) layer that permits the flow of water vapor and air for activating the exothermic reaction, but prevents an excessive amount of liquids from contacting the exothermic composition, which could either suppress the reaction or result in an excessive amount of heat that overly warms or burns the user. It should be understood that numerous other possible combinations and configurations would be well within the ordinary skill of those in the art. Various configuration for other configurations for warming products are described, for instance, in U.S. Patent Application Publication Nos. 2006/0141882 to Quincy, et al.; 2006/0142828 to Schorr, et al.; 2007/0142882 to Quincy, et al.; 2007/0142883 to Quincy; 2007/0156213 to Friedensohn, et al.; and 2007/0141929 to Quincy, et al., all of which are incorporated herein in their entirety by reference thereto for all relevant purposes.

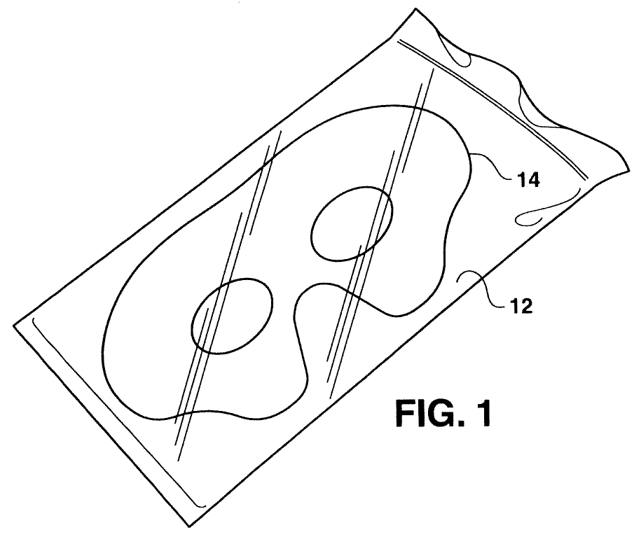

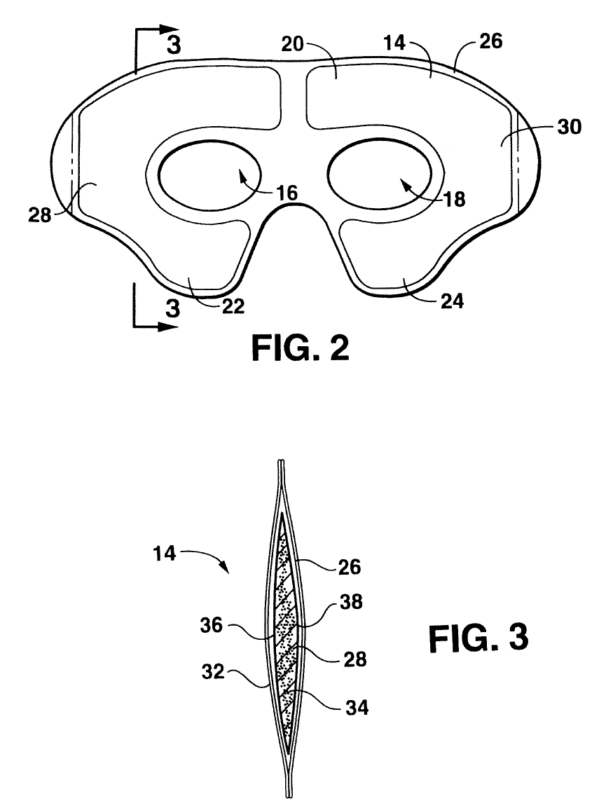

The warming product may be configured for placement on the skin (e.g., wrapped around) or it may define an interior into which a user may insert a portion of his or her body. Further, the warming product may have any desired shape or size to accommodate its use on a body part, such as the face, finger, toe, hand, foot, wrist, forearm, etc. Referring to FIG. 1, one embodiment of a warming product 14 is shown in the shape of a mask sealed within a package 12 that inhibits exposure of the mask 14 to ambient air prior to activation. FIG. 2 illustrates the mask 14 after removal from the package 12. The shape of the mask may depend upon the intended use of the mask. For instance, a mask designed for therapeutic spa-like benefits may have a different shape than a mask used to treat sinus infections. In fact, the mask can be designed to cover the entire face, neck and chest of a user. In an alternative embodiment, the mask can be designed to cover a relatively small portion of a person's face. In the embodiment illustrated in FIG. 2, the mask is designed to cover a person's forehead and to surround the eyes and nose of a user. In this regard, the mask 14 includes a first eye opening 16 spaced from a second eye opening 18. The mask 14 further includes a forehead portion 20 located above the eye openings 16 and 18. In addition, the mask 14 includes a pair of lobes that extend downwardly. Specifically, the mask includes a first cheek portion 22 that extends downwardly from the first eye opening 16 and a second cheek portion 24 that extends downwardly from the second eye opening 18. The cheek portions 22 and 24 are designed to surround the nose of a user.

When the mask 14 is designed to treat a person suffering from congestion, sinus pressure and pain, it is generally desirable that the mask does not surround the nose of a user so that a user can continue to blow his or her nose even while wearing the mask. For instance, as shown in FIG. 2, the mask 14 may include an access area for the nostrils. In other applications, however, the mask may also include a nose portion that also covers the nose of a user. The nose portion may contain an elastic material, a gathered material that has sufficient slack to go over the nose of a user, or may project outwardly from the mask so that the nose can fit comfortably below the mask. The mask may include a nose portion, for instance, when it is desirable to apply heat directly to the nose of a user, such as during a spa application or perhaps to provide pain relief when the nose has been injured.

The mask 14 of FIG. 2 also includes a facing layer 26 that supports a first warming pad 28 and a second warming pad 30. Although the embodiment in FIG. 2 shows first and second warming pads 28 and 30, it should be understood that more or less delivery pads may be present. For instance, in one embodiment, the mask may include a single warming pad that is generally in the shape of the entire mask. The first warming pad 28 partially encircles the eye opening 16 and thus extends into the forehead portion 20 and down into the first cheek portion 22. Similarly, the second warming pad 30 partially encircles the second eye opening 18 and also delivers heat to the forehead portion 20 and to the second cheek portion 24. In this manner, heat is provided to a user around the eyes, over the forehead, and adjacent to the nose.

Referring to FIG. 3, the construction of the warming pad 28 is shown in more detail. As illustrated, the warming pad 28 includes an exothermic composition 34 sandwiched and sealed in between a first polymer film 36 and a second polymer film 38. The second polymer film 38 is positioned to face a user and to deliver heat. To activate the exothermic composition 34, air enters the warming pad through at least one of the polymer films. In this regard, at least a portion of the warming pad 28 includes a gas permeable portion. For instance, in one embodiment, at least a portion of the polymer film 36 is gas permeable or breathable, while remaining impermeable to liquids.

The warming pads 28 and 30 are attached to a facing layer 26 using any known technique, such as thermally bonding, ultrasonically bonding, adhesive bonding, etc. The facing layer 26 may be constructed from nonwoven webs, woven fabrics, knit fabrics, paper webs, etc. Although optional, the mask can further include the outer cover layer 32 to improve the aesthetics and better hold the warming pads in position. For instance, the outer cover layer can be bonded to the facing layer 26 in a manner that forms pockets for the warming pads. The outer cover layer 32 has sufficient gas permeability so as not to interfere with the ability of the warming pad to receive air for gas diffusion. Thus, if present, the outer cover layer can comprise a nonwoven web having a relatively light basis weight and significant porosity.

To hold the mask 14 onto the face of a user, the mask can include a strap (not shown) applied to the facing layer 26. The strap can be made from any suitable material. In one embodiment, for instance, the strap is formed from an elastic material. For instance, the strap can be made from an elastic film or an elastic laminate. In one embodiment, for instance, the strap can be made from a stretch bonded laminate or from a neck bonded laminate. Such materials may provide comfort to the user. Besides a strap, the mask can also include an adhesive for attaching the mask to a person's face.

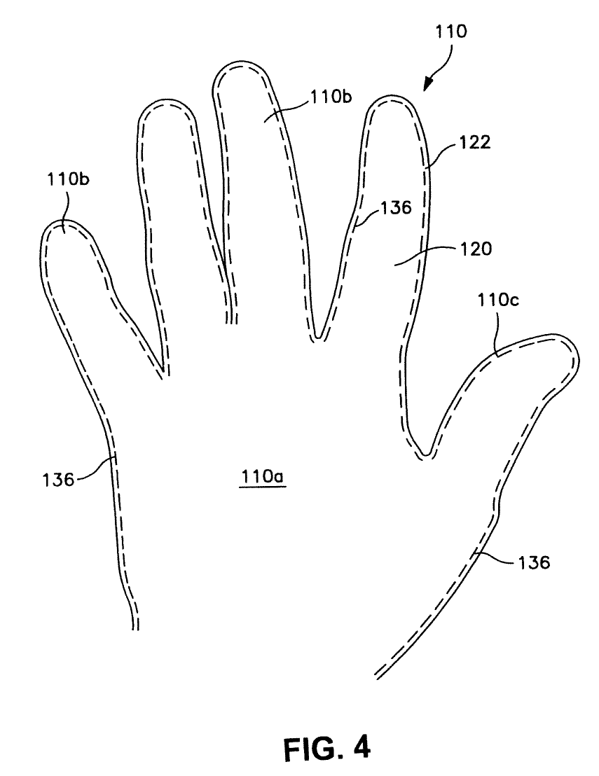

In addition to a mask, the warming product of the present invention may also have a variety of other configurations, such as gloves, socks, sleeves, mittens, etc. Referring to FIG. 4, for instance, one embodiment of a glove 110 is shown that is in the shape of a human hand. The glove 110 has a palm region 110a, a plurality of finger portions 110b, and a thumb portion 110c. In this particular embodiment, the glove 110 contains substrates 120 and 122 that are joined at a location proximate to their perimeters by sewing and then inverting the glove 110 so that a seam 136 becomes located on the interior of the glove 110. Of course, the glove 110 need not be inverted, and the seam 136 can remain on the exterior of the glove 110. Also, the substrates 120 and 122 need not be joined in a way that produces a seam. For example, the edges of the substrates 120 and 122 may be placed adjacent to each other and joined ultrasonically, thermally, adhesively, cohesively, using tape, by fusing the materials together (e.g., by using an appropriate solvent), by welding the materials together, or by other approaches.

Regardless of the particular configuration of the warming product, a heating profile may be achieved in which an elevated temperature is reached quickly and maintained over an extended period of time. For example, a temperature increase of at least about 1° C., in some embodiments at least about 2° C., and in some embodiments, at least about 3° C., may be achieved in about 1 hour or less, in some embodiments about 30 minutes or less, and in some embodiments, from about 0.1 to about 15 minutes. This may result in an elevated temperature of from about 30° C. to about 60° C., in some embodiments from about 32° C. to about 55° C., and in some embodiments from about 34° C. to about 50° C. This elevated temperature may be substantially maintained for at least about 1 hour, in some embodiments at least about 2 hours, in some embodiments at least about 4 hours, and in some embodiments, at least about 10 hours (e.g., for overnight use). The amount of time that the warming product remains heated can depend upon the particular application. For instance, when the product is used for aroma therapy or for use in spa-like therapeutic applications, it may only need to be heated for a period of time of about 15 minutes. When treating a user for sinus congestion, the common cold, or for pain relief, however, the product may remain heated for a time of from about 1 hour to about 6 hours, such as from about 2 hours to about 5 hours.

When the warming product reaches the desired elevated temperature, the thermochromic composition of the present invention may possess one color that indicates to the user that the product is functioning. When the exothermic reactants are exhausted and the warming product begins to cool, however, the thermochromic composition undergoes a color change to indicate to the user that the treatment is complete or near completion. This color change is rapid and may be readily detected within a relatively short period of time. For example, a visual change in color may occur in about 30 seconds or less, in some embodiments about 15 seconds or less, and in some embodiments, about 5 seconds or less. Further, the visual color change may remain observable for a sufficient length of time, such as about 1 second or more, in some embodiments about 2 seconds or more, and in some embodiments, from about 3 seconds to about 1 minute. The extent of the color change, which may be determined either visually or using instrumentation (e.g., optical reader), is also generally sufficient to provide a “real-time” indication. This color change may, for example, be represented by a certain change in the absorbance reading as measured using a conventional test known as “CIELAB”, which is discussed in Pocket Guide to Digital Printing by F. Cost, Delmar Publishers, Albany, N.Y. ISBN 0-8273-7592-1 at pages 144 and 145. This method defines three variables, L*, a*, and b*, which correspond to three characteristics of a perceived color based on the opponent theory of color perception. The three variables have the following meaning:

L*=Lightness (or luminosity), ranging from 0 to 100, where 0=dark and 100=light;

a*=Red/green axis, ranging approximately from −100 to 100; positive values are reddish and negative values are greenish; and

b*=Yellow/blue axis, ranging approximately from −100 to 100; positive values are yellowish and negative values are bluish.

Because CIELAB color space is somewhat visually uniform, a single number may be calculated that represents the difference between two colors as perceived by a human. This difference is termed ΔE and calculated by taking the square root of the sum of the squares of the three differences (ΔL*, Δa*, and Δb*) between the two colors. In CIELAB color space, each ΔE unit is approximately equal to a “just noticeable” difference between two colors. CIELAB is therefore a good measure for an objective device-independent color specification system that may be used as a reference color space for the purpose of color management and expression of changes in color. Using this test, color intensities (L*, a*, and b*) may thus be measured using, for instance, a handheld spectrophotometer from Minolta Co. Ltd. of Osaka, Japan (Model # CM2600d). This instrument utilizes the D/8 geometry conforming to CIE No. 15, ISO 7724/1, ASTME1164 and JIS Z8722-1982 (diffused illumination/8-degree viewing system. The D65 light reflected by the specimen surface at an angle of 8 degrees to the normal of the surface is received by the specimen-measuring optical system. Typically, the color change that results is represented by a ΔE of about 2 or more, in some embodiments about 3 or more, and in some embodiments, from about 5 to about 50.

The present invention may be better understood with reference to the following example.

### EXAMPLE

The ability to incorporate a thermochromic composition into a nonwoven fabric for incorporation into a warming product was demonstrated. More specifically, coform nonwoven fabrics (basis weight of 61 grams per square meter) were made by mixing thermochromic pigment/polypropylene concentrated pellets (Chromicolor concentrates, Matsui International Co. Inc., Gardena Calif.) with meltblown-grade polypropylene pellets (PF-015 Exxon) in a cement mixer for 10 minutes. This effectively blended down the pigment concentration from 18% down to 3.6% wt/wt. The resin mix was then spun into a meltblown that had cellulose fibers blown into the fiber cascade. The following conditions were employed:

| | |
|---|---|
| Extruder melt temperature: | 460° F. |
| Extruder melt pressure: | 260 psi |
| Polymer pipe temperature: | 500° F. |
| Polymer hose temperature: | 500° F. |
| Spin pump temperature: | 500° F. |
| Spin pump pressure: | 502 psi |
| Spin pump speed: | 12 rpms |
| Die melt temperature: | 491/522° F. |
| Die pressure: | 70 psi |
| Die primary air temperature: | 700° F. |
| Die primary air pressure at tip: | 2.0 psi |
| Die heater: | 500° F. |
| Picker spped: | 2650 rpms |
| Die height: | 12 inches |
| Pulp nozzle height: | 16 inches |
| Die tip angle: | 45° |
| Line speed: | 225 fpm |

Extruder zone 1=260° F., zone 2=370° F., zone 3=460° F.; zone 4=480° F., zone 5=500° F., zone 6=500° F.

The resulting fabric contained a 70:30 weight ratio of polypropylene:cellulose. The thermochromic pigments employed were either blue to white (41° C. temperature transition, HQ type#37 grid G#0, turquoise blue) or green to orange (33° C. temperature transition, I2, type#27 brilliant green to orange), both obtained from Matsui International Co. Inc. (Gardena Calif.).

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A warming product for providing heat treatment to the body part of a user, the warming product comprising:
an exothermic composition that releases heat for a certain time period upon activation, the time period defining a heating cycle;
a thermochromic composition that possesses a first color during the heating cycle and a second color after completion of the heating cycle, the first color being visually distinguishable from the second color.

2. The warming product of claim 1, wherein the thermochromic composition undergoes a color change at a temperature of from about 30° C. to about 60° C.

3. The warming product of claim 1, wherein the thermochromic composition undergoes a color change at a temperature of from about 34° C. to about 50° C.

4. The warming product of claim 1, wherein the thermochromic composition includes liquid crystals.

5. The warming product of claim 1, wherein the thermochromic composition includes microcapsules that contain a proton-accepting chromogen and a desensitizer, wherein the desensitizer possesses a melting point above which the chromogen is capable of becoming protonated, thereby resulting in a color change.

6. The warming product of claim 5, wherein the proton-accepting chromogen is a leuco dye.

7. The warming product of claim 5, wherein the desensitizer has a boiling point of about 150° C. or higher and a melting point of about from about 30° C. to about 60° C.

8. The warming product of claim 5, wherein the microcapsules further comprise a proton-donating agent.

9. The warming product of claim 1, wherein the warming product contains a substrate.

10. The warming product of claim 9, wherein the thermochromic composition is disposed on a surface of the substrate.

11. The warming product of claim 9, wherein the thermochromic composition is incorporated into the substrate.

12. The warming product of claim 11, wherein the exothermic composition is disposed on a surface of the substrate.

13. The warming product of claim 9, wherein the substrate contains a nonwoven web.

14. The warming product of claim 1, wherein the exothermic composition contains an oxidizable metal that undergoes an exothermic reaction in the presence of moisture and air.

15. The warming product of claim 14, wherein the exothermic composition further comprises a carbon component and a binder.

16. The warming product of claim 1, wherein the warming product is a mask.

17. The warming product of claim 1, wherein the warming product is a glove.

18. A method for monitoring the degree of heat being provided to a body part of a user, the method comprising:
providing a warming product that comprises comprising an exothermic composition that releases heat for a certain time period upon activation, the time period defining a heating cycle, the warming product further comprising a thermochromic composition that possesses a first color during the heating cycle and a second color after completion of the heating cycle;
activating the exothermic composition;
placing the warming product adjacent to a body part; and
observing the thermochromic composition, wherein observation of the first color indicates that the exothermic composition is releasing heat during the heating cycle and observation of the second color indicates that the heating cycle is complete.

19. The method of claim 18, wherein the thermochromic composition undergoes a color change at a temperature of from about 30° C. to about 60° C.

20. The method of claim 18, wherein the thermochromic composition undergoes a color change at a temperature of from about 34° C. to about 50° C.

21. The method of claim 18, wherein the thermochromic composition includes microcapsules that contain a proton-accepting chromogen and a desensitizer, wherein the desensitizer possesses a melting point above which the chromogen is capable of becoming protonated, thereby resulting in a color change.

22. The method of claim 18, wherein the exothermic composition contains an oxidizable metal that undergoes an exothermic reaction in the presence of moisture and air.

23. The method of claim 22, wherein the warming product is initially sealed within an enclosure that inhibits the passage of oxygen to the exothermic composition, the activation step including opening the enclosure and exposing the warming product to air.

24. The method of claim 18, wherein the body party is the face.

25. The method of claim 18, wherein the body part is a hand or foot.
